(19) United States
(12) Patent Application Publication
(10) Pub. No.: US 2015/0297674 A1
(43) Pub. Date: Oct. 22, 2015

(54) SUBSTANCES AND METHODS FOR THE TREATMENT OF CEREBRAL AMYLOID ANGIOPATHY RELATED CONDITIONS OR DISEASE

(71) Applicant: Loma Linda University Medical Center, Loma Linda, CA (US)

(72) Inventors: Wolff M. Kirsch, Redlands, CA (US); Andrew Crofton, Loma Linda, CA (US); Tanya Freeman, Loma Linda, CA (US); Charles Glabe, Irvine, CA (US); SAmuel M. Hudson, Raleigh, NC (US); Matthew Schrag, San Bernardino, CA (US); Harry Vinters, Los Angeles, CA (US); Matthew Zabel, Loma Linda, CA (US)

(21) Appl. No.: 14/382,402

(22) PCT Filed: Mar. 12, 2013
(86) PCT No.: PCT/US2013/030582

§ 371 Date: Sep. 2, 2014

Related U.S. Application Data
(60) Provisional application No. 61/609,816, filed on Mar. 12, 2012.

(51) Int. Cl. A61K 38/17; A61K 47/48

(52) U.S. Cl. CPC A61K 38/177; A61K 47/48923; A61K 47/48723

(57) ABSTRACT

A substance for treating a cerebral amyloid angiopathy related condition or disease affecting cerebrovasculature in a patient, comprising an inhibitor that causes inhibition of the formation of membrane attack complex of the complement system; and a vehicle for transporting the inhibitor into the cerebrovasculature; where the inhibition by the inhibitor is sufficient to decrease the incidence of or to prevent the incidence of cytolysis of the smooth muscle cells. A method for treating a cerebral amyloid angiopathy related condition or disease affecting cerebrovasculature in a patient, comprising: a) identifying a patient with a cerebral amyloid angiopathy related condition or disease; b) providing one or more than one substance that comprises an inhibitor that causes inhibition of the formation of membrane attack complex of the complement system, c) administering one or more than one dose of the one or more than one substance to the patient.

### CROSS-REFERENCE TO RELATED APPLICATIONS

The present Application claims the benefit of U.S. Provisional Patent Application 61/609,816, titled “Method and Substance for the Treatment of Cerebral Amyloid Angiopathy,” filed Mar. 12, 2012, the contents of which are incorporated in this disclosure by reference in their entirety.

### STATEMENT REGARDING FEDERALLY SPONSORED RESEARCH OR DEVELOPMENT

This invention was made with United States Government support under Grant No. 5 R01 AG020948, awarded by the National Institutes of Health/National Institute on Aging. The United States Government has certain rights in this invention.

### BACKGROUND

Cerebral amyloid angiopathy (CAA) is a nonspecific disease entity that has been associated with a number of neuropathologic conditions, including a 70-90% prevalence in patients diagnosed with Alzheimer's disease (AD). Cerebral amyloid angiopathy is characterized by the pathologic accumulation of beta-amyloid (amyloid B, AB) plaques in the tunica media and tunica adventitia of small and mid-sized arteries (and less frequently of veins) of the cerebral cortex and the leptomeninges resulting in vascular fragility, intracranial bleeding (lobar intracerebral hemorrhage) and in some cases dementia.

Currently, there is no known effective treatment to decrease or prevent the underlying deposition of beta-amyloid that characterizes cerebral amyloid angiopathy. Though beta-amyloid immunotherapy rapidly clears amyloid plaques, cerebral amyloid angiopathy is worsened with increased brain inflammation and increased brain microbleeds. Therefore, the current goal of treatment is symptomatic, and includes physical rehabilitation and amelioration of seizures when present.

Therefore, there is a need for a method for treating cerebral amyloid angiopathy.

### SUMMARY

According to one embodiment of the present invention, there is provided a substance for treating a cerebral amyloid angiopathy related condition or disease affecting cerebrovasculature in a patient, where the cerebrovasculature comprises tunica intima comprising endothelial cells, tunica media comprising smooth muscle cells, and tunica adventitia, where the condition or disease is associated with an incidence of cytolysis of the smooth muscle cells from beta-amyloid deposition in the cerebrovasculature that leads to formation of membrane attack complex of the complement system in the smooth muscle cells. The substance comprises one or more than one inhibitor that causes inhibition of the formation of membrane attack complex of the complement system, and one or more than one vehicle for transporting the one or more than one inhibitor into the cerebrovasculature, where the inhibition by the inhibitor is sufficient to decrease the incidence of or to prevent the incidence of cytolysis of the smooth muscle cells. In one embodiment, the substance crosses the tunica intima and enters the tunica media. In one embodiment, the patient has a blood brain barrier comprising the tunica intima, the tunica media and the tunica adventitia, and where the substance crosses the blood brain barrier. In one embodiment, the one or more than one inhibitor is a plurality of inhibitors. In another embodiment, the plurality of inhibitors is two inhibitors. In another embodiment, the plurality of inhibitors is three inhibitors. In another embodiment, the plurality of inhibitors is four inhibitors. In one embodiment, the cerebral amyloid angiopathy related condition or disease is selected from the group consisting of one or more than one of Alzheimer's disease, a brain microbleed, cerebral amyloidosis of the parenchyma, mild cognitive impairment with amyloid plaques in the brain and a combination of the preceding. In one embodiment, the one or more than one inhibitor specifically causes inhibition of the formation of membrane attack complex of the complement system in the tunica intima of the cerebrovasculature, tunica media of the cerebrovasculature, or both the tunica intima of the cerebrovasculature and tunica media of the cerebrovasculature. In one embodiment, one or more than one of the one or more than one inhibitor that causes inhibition of the formation of membrane attack complex of the complement system upregulates CD59 glycoprotein levels in the cerebrovasculature of the patient. In one embodiment, one or more than one of the one or more than one inhibitor that causes inhibition of the formation of membrane attack complex of the complement system is selected from the group consisting of one or more than one of alphaGal lectin, anti-C5 Mab, C1-Inhibitor, factor H, human CD59 cDNA, and a combination of the preceding. In one embodiment, one of the one or more than one inhibitor that causes inhibition of the formation of membrane attack complex of the complement system is a plasmid comprising human CD59 cDNA. In one embodiment, one of the one or more than one inhibitor comprises human CD59 cDNA in the pCMV6-AC plasmid, or human CD59 cDNA in the pCMV6-XL5 plasmid. In one embodiment, the one or more than one vehicle is a plurality of vehicles. In another embodiment, the plurality of vehicles is two vehicles. In another embodiment, the plurality of vehicles is three vehicles. In another embodiment, the plurality of vehicles is four vehicles. In one embodiment, one or more than one of the one or more than one vehicle is selected from the group consisting of chitosan nanoparticles, colloidal metallic nanoparticles, polymer nanoparticles and viral particles. In one embodiment, at least one of the one or more than one vehicle comprises chitosan nanoparticles. In one embodiment, the substance comprises one vehicle and a plurality of inhibitors. In another embodiment, the substance comprises one vehicle and two inhibitors. In one embodiment, the substance further comprises one or more than one targeting agent that recognizes the beta-amyloid deposited in the cerebrovasculature, where one or more than one targeting agent forms at least part of the surface of the substance when one or more than one targeting agent is combined with the inhibitor and the vehicle, thereby directing the substance to the beta-amyloid deposited in the cerebrovasculature when the substance is administered to the patient. In one embodiment, the targeting agent recognizes and attaches to a subset of conformationally unique beta-amyloid deposited in the cerebrovasculature, where the conformationally unique beta-amyloid deposited in the cerebrovasculature is specific for cerebral amyloid angiopathy. In one embodiment, when combined with the inhibitor and the vehicle, the targeting agent directs the substance to the beta-amyloid deposited in the cerebrovascular smooth muscle cells when the substance is administered to the patient. In one embodiment, at least one of the one or more than one targeting agent is a monoclonal antibody or is a fragment of a monoclonal antibody. In one embodiment, at least one of the one or more than one targeting agent is selected from the group consisting of amyloid antibody (M31) Fab fragments, 6E10 beta amyloid monoclonal antibody and a combination of the preceding. In one embodiment, the one or more than one targeting agent is a plurality of targeting agents. In another embodiment, the plurality of targeting agents is two targeting agents. In another embodiment, the plurality of targeting agents is three targeting agents. In another embodiment, the plurality of targeting agents is four targeting agents. In one embodiment, the substance further comprises one or more than one additional chemical that enables determination of plasmid transfection efficacy where the inhibitor is a plasmid, or enables tracking of the substance. In one embodiment, at least one of the one or more than one additional chemical is selected from the group consisting of hydroxycoumarin and green fluorescent protein.

According to another embodiment of the present invention, there is provided a pharmaceutical for treating a cerebral amyloid angiopathy related condition or disease. The pharmaceutical comprises one or more than one substance according to the present invention, and further comprises one or more than one of a binder, a buffer, a coloring chemical, a flavoring chemical and a preservative.

According to another embodiment of the present invention, there is provided a method for treating a cerebral amyloid angiopathy related condition or disease affecting cerebrovasculature in a patient, where the cerebrovasculature comprises tunica intima comprising endothelial cells, tunica media comprising smooth muscle cells, and tunica adventitia, where the condition or disease is associated with an incidence of cytolysis of the smooth muscle cells from beta-amyloid deposition in the cerebrovasculature that leads to formation of membrane attack complex of the complement system in the smooth muscle cells. The method comprises a) identifying a patient with a cerebral amyloid angiopathy related condition or disease suitable for treatment; b) providing one or more than one substance that comprises an inhibitor that causes inhibition of the formation of membrane attack complex of the complement system, or comprises providing one or more than one pharmaceutical comprising one or more than one substance that comprises an inhibitor that causes inhibition of the formation of membrane attack complex of the complement system, or comprises providing both one or more than one substance that comprises an inhibitor that causes inhibition of the formation of membrane attack complex of the complement system and one or more than one pharmaceutical comprising one or more than one substance that comprises an inhibitor that causes inhibition of the formation of membrane attack complex of the complement system, where the inhibition by the inhibitor is sufficient to decrease the incidence of or to prevent the incidence of cytolysis of the smooth muscle cells; and c) administering one or more than one dose of the one or more than one substance or administering one or more than one dose of the one or more than one pharmaceutical to the patient by a route. In one embodiment, the patient is a human. In another embodiment, the cerebral amyloid angiopathy related condition or disease is selected from the group consisting of one or more than one of Alzheimer's disease, a brain microbleed, cerebral amyloidosis of the parenchyma, mild cognitive impairment with amyloid plaques in the brain and a combination of the preceding. In one embodiment, identifying the patient comprises consulting patient records to determine if the patient has a cerebral amyloid angiopathy related condition or disease suitable for treatment. In one embodiment, identifying the patient comprises diagnosing the patient with a cerebral amyloid angiopathy related condition or disease suitable for treatment. In a preferred embodiment, diagnosing the patient comprises performing one or more than one of action selected from the group consisting of identifying one or more than one marker for cerebral amyloid angiopathy in blood or another body fluid of the patient, performing cognitive testing, performing an invasive procedure, performing a non-invasive imaging procedure, and performing a physical examination. In one embodiment, one or more than one of the one or more than one substance is a substance according to the present invention. In one embodiment, one or more than one of the one or more than one pharmaceutical is a pharmaceutical according to the present invention. In one embodiment, the one or more than one substance is a plurality of substances. In another embodiment, the one or more than one substance is two substances. In one embodiment, the one or more than one pharmaceutical is a plurality of pharmaceuticals. In another embodiment, the one or more than one pharmaceutical is two pharmaceuticals. In one embodiment, the one or more than one dose is one dose. In another embodiment, the one or more than one dose is a plurality of doses. In another embodiment, the plurality of doses is two doses. In another embodiment, the plurality of doses is three doses. In another embodiment, the plurality of doses is four doses. In another embodiment, the plurality of doses is more than four doses. In one embodiment, the one or more than one dose is administered daily for a predetermined amount of time. In another embodiment, the one or more than one dose is administered twice daily for a predetermined amount of time. In another embodiment, the one or more than one dose is administered weekly for a predetermined amount of time. In another embodiment, the one or more than one dose is administered monthly for a predetermined amount of time. In another embodiment, the one or more than one dose is administered between once a day and once a week for a predetermined amount of time. In another embodiment, the one or more than one dose is administered between once a day and once a month for a predetermined amount of time. In another embodiment, the dose is between 0.000001 mg/m² body surface area and 100 g/m² body surface area. In another embodiment, the dose is between 0.0001 mg/m² body surface area and 10 g/m² body surface area. In another embodiment, the dose is between 1 mg/m² body surface area and 1 g/m² body surface area. In one embodiment, the route is selected from the group consisting of intraarterial injection, intramuscular injection, intranasal spray and intravenous injection. In one embodiment, the method further comprises determining the effect of treatment on the patient. In one embodiment, determining the effect of treatment on the patient comprises performing one or more than one of action selected from the group consisting of identifying one or more than one marker for cerebral amyloid angiopathy in blood or another body fluid of the patient, performing cognitive testing, performing an invasive procedure, performing a non-invasive imaging procedure, and performing a physical examination. In one embodiment, the method further comprises adjusting treatment. In another embodiment, adjusting treatment comprises administering to the patient one or more than one additional dose of the one or more than one substance or administering one or more than one additional dose of the one or more than one pharmaceutical to the patient. In one embodiment, the substance further comprises one or more than one vehicle for transporting the one or more than one inhibitor into the cerebrovasculature. In one embodiment, the substance crosses the tunica intima and enters the tunica media. In one embodiment, the patient has a blood brain barrier comprising the tunica intima, the tunica media and the tunica adventitia, and where the substance crosses the blood brain barrier. In one embodiment, the one or more than one inhibitor is a plurality of inhibitors. In another embodiment, the plurality of inhibitors is two inhibitors. In another embodiment, the plurality of inhibitors is three inhibitors. In another embodiment, the plurality of inhibitors is four inhibitors. In one embodiment, the one or more than one inhibitor specifically causes inhibition of the formation of membrane attack complex of the complement system in the tunica intima of the cerebrovasculature, tunica media of the cerebrovasculature, or both the tunica intima of the cerebrovasculature and tunica media of the cerebrovasculature. In one embodiment, one or more than one of the one or more than one inhibitor that causes inhibition of the formation of membrane attack complex of the complement system upregulates CD59 glycoprotein levels in the cerebrovasculature of the patient. In one embodiment, one or more than one of the one or more than one inhibitor that causes inhibition of the formation of membrane attack complex of the complement system is selected from the group consisting of one or more than one of alphaGal lectin, anti-C5 Mab, C1-Inhibitor, factor H, human CD59 cDNA, and a combination of the preceding. In one embodiment, one of the one or more than one inhibitor that causes inhibition of the formation of membrane attack complex of the complement system is a plasmid comprising human CD59 cDNA. In one embodiment, one of the one or more than one inhibitor comprises human CD59 cDNA in the pCMV6-AC plasmid, or human CD59 cDNA in the pCMV6-XL5 plasmid. In one embodiment, the one or more than one vehicle is a plurality of vehicles. In another embodiment, the plurality of vehicles is two vehicles. In another embodiment, the plurality of vehicles is three vehicles. In another embodiment, the plurality of vehicles is four vehicles. In one embodiment, the one or more than one vehicle crosses the tunica intima and enters the tunica media. In another embodiment, the patient has a blood brain barrier comprising the tunica intima, the tunica media and the tunica adventitia, and the one or more than one vehicle crosses the blood brain barrier. In one embodiment, one or more than one of the one or more than one vehicle is selected from the group consisting of chitosan nanoparticles, colloidal metallic nanoparticles, polymer nanoparticles and viral particles. In another embodiment, at least one of the one or more than one vehicle comprises chitosan nanoparticles. In another embodiment, the substance comprises one vehicle and a plurality of inhibitors. In another embodiment, the substance comprises one vehicle and two inhibitors. In another embodiment, the method further comprises one or more than one targeting agent that recognizes the beta-amyloid deposited in the cerebrovasculature, where one or more than one targeting agent forms at least part of the surface of the substance when one or more than one targeting agent is combined with the inhibitor and the vehicle, thereby directing the substance to the beta-amyloid deposited in the cerebrovasculature when the substance is administered to the patient. In one embodiment, the targeting agent recognizes and attaches to a subset of conformationally unique beta-amyloid deposited in the cerebrovasculature, where the conformationally unique beta-amyloid deposited in the cerebrovasculature is specific for cerebral amyloid angiopathy. In one embodiment, when combined with the inhibitor and the vehicle, the targeting agent directs the substance to the beta-amyloid deposited in the cerebrovascular smooth muscle cells when the substance is administered to the patient. In one embodiment, at least one of the one or more than one targeting agent is a monoclonal antibody or is a fragment of a monoclonal antibody. In one embodiment, at least one of the one or more than one targeting agent is selected from the group consisting of amyloid antibody (M31) Fab fragments, 6E10 beta amyloid monoclonal antibody and a combination of the preceding. In one embodiment, the one or more than one targeting agent is a plurality of targeting agents. In another embodiment, the plurality of targeting agents is two targeting agents. In another embodiment, the plurality of targeting agents is three targeting agents. In another embodiment, the plurality of targeting agents is four targeting agents. In one embodiment, the substance further comprises one or more than one additional chemical that enables determination of plasmid transfection efficacy where the inhibitor is a plasmid, or enables tracking of the substance. In one embodiment, at least one of the one or more than one additional chemical is selected from the group consisting of hydroxycoumarin and green fluorescent protein. In one embodiment, the pharmaceutical further comprises one or more than one of a binder, a buffer, a coloring chemical, a flavoring chemical and a preservative.

### DRAWINGS

These and other features, aspects and advantages of the present invention will become better understood with regard to the following description, appended claims, and accompanying drawings where:

FIG. 1 is a schematic depiction of one embodiment of the human CD59 cDNA in the pCMV6-AC useful in the present invention;

FIG. 2 shows western blots showing the accumulation of C6 and beta-actin (a protein expressed in all cells and used as a loading control to normalize C6 values) in blood vessel walls from control brain tissue (left), in blood vessel walls from brain tissue from patients diagnosed with Alzheimer's disease (AD, center), and in blood vessel walls from brain tissue from patients diagnosed with Alzheimer's disease concurrent with cerebral amyloid angiopathy (AD/CAA, right);

FIG. 3 is a histogram showing the average concentration of C6 from multiple western blots (n=4) in blood vessel walls from control brain tissue, in blood vessel walls from brain tissue from patients diagnosed with Alzheimer's disease (AD), and in blood vessel walls from brain tissue from patients diagnosed with Alzheimer's disease concurrent with cerebral amyloid angiopathy (AD/CAA);

FIG. 4 are photomicrographs showing C6 localization in the walls of blood vessels from control brain tissue (left), in the walls of blood vessels from brain tissue from patients diagnosed with Alzheimer's disease (AD, center), and in the walls of blood vessels from brain tissue from patients diagnosed with Alzheimer's disease concurrent with cerebral amyloid angiopathy (AD/CAA, right) by immunohisto-chemistry with DAB-staining; and

FIG. 5 are confocal micrographs of microglia walls from control brain tissue (A, left), blood vessel walls from brain tissue from patients diagnosed with Alzheimer's disease (B, center), and blood vessel walls from brain tissue from patients diagnosed with Alzheimer's disease concurrent with cerebral amyloid angiopathy (C, right), where the brain tissue was stained for alpha2-macroglobulin and beta-amyloid (A, B), and for beta-amyloid and CD11b (C).

### DESCRIPTION

According to one embodiment of the present invention, there is provided a substance for treating a cerebral amyloid angiopathy related condition or disease affecting cerebrovasculature in a patient, where the cerebrovasculature comprises tunica intima comprising endothelial cells, tunica media comprising smooth muscle cells, and tunica adventitia, where the condition or disease is associated with an incidence of cytolysis of the smooth muscle cells from beta-amyloid deposition in the cerebrovasculature that leads to formation of membrane attack complex of the complement system in the smooth muscle cells, where the substance comprises one or more than one inhibitor that causes inhibition of the formation of membrane attack complex of the complement system, and where the one or more than one inhibition by the inhibitor is sufficient to decrease the incidence of or to prevent the incidence of cytolysis of the smooth muscle cells. In one embodiment, the substance further comprises a vehicle and a targeting agent. According to another embodiment of the present invention, there is provided a pharmaceutical for treating a cerebral amyloid angiopathy related condition or disease. The pharmaceutical comprises a substance according to the present invention. According to another embodiment of the present invention, there is provided a method for treating a cerebral amyloid angiopathy related condition or disease affecting cerebrovasculature in a patient, where the cerebrovasculature comprises tunica intima comprising endothelial cells, tunica media comprising smooth muscle cells, and tunica adventitia, where the condition or disease is associated with an incidence of cytolysis of the smooth muscle cells from beta-amyloid deposition in the cerebrovasculature that leads to formation of membrane attack complex of the complement system in the smooth muscle cells. The method comprises providing one or more than one substance that comprises an inhibitor that causes inhibition of the formation of membrane attack complex of the complement system, or comprises providing one or more than one pharmaceutical comprising one or more than one substance that comprises an inhibitor that causes inhibition of the formation of membrane attack complex of the complement system, or comprises providing both one or more than one substance that comprises an inhibitor that causes inhibition of the formation of membrane attack complex of the complement system and one or more than one pharmaceutical comprising one or more than one substance that comprises an inhibitor that causes inhibition of the formation of membrane attack complex of the complement system, where the inhibition by the inhibitor is sufficient to decrease the incidence of or to prevent the incidence of cytolysis of the smooth muscle cells, and administering one or more than one dose of one or more than one substance or of the one or more than one pharmaceutical to the patient by a route. In one embodiment, one or more than one of the one or more than one substance is a substance according to the present invention. In another embodiment, one or more than one of the one or more than one pharmaceutical is a pharmaceutical according to the present invention. In one embodiment, the cerebral amyloid angiopathy related condition or disease is selected from the group consisting of one or more than one of Alzheimer's disease, a brain microbleed, cerebral amyloidosis of the parenchyma, mild cognitive impairment with amyloid plaques in the brain and a combination of the preceding. The substances, methods and pharmaceuticals will now be disclosed in detail.

As used in this disclosure, except where the context requires otherwise, the term “comprise” and variations of the term, such as “comprising,” “comprises” and “comprised” are not intended to exclude other additives, components, integers or steps.

As used in this disclosure, except where the context requires otherwise, the method steps disclosed are not intended to be limiting nor are they intended to indicate that each step is essential to the method or that each step must occur in the order disclosed.

As used in this disclosure, except where the context requires otherwise, the term “cerebrovasculature” and related terms refer to both cerebral vasculature and leptomeningeal vasculature.

As used in this disclosure, except where the context requires otherwise, “cerebral amyloid angiopathy related condition or disease” means “cerebral amyloid angiopathy” and any condition or disease that includes “cerebral amyloid angiopathy” as a component of the condition or disease, such as for example Alzheimer's disease, a brain microbleed, cerebral amyloidosis of the parenchyma, mild cognitive impairment with amyloid plaques in the brain. Further, “cerebral amyloid angiopathy related condition or disease” means “one or more than one cerebral amyloid angiopathy related condition, one or more than one cerebral amyloid angiopathy related disease, or both one or more than one cerebral amyloid angiopathy related condition and one or more than one cerebral amyloid angiopathy related disease.”

According to one embodiment of the present invention, there is provided a substance for treating a cerebral amyloid angiopathy related condition or disease affecting cerebrovasculature in a patient, where the cerebrovasculature comprises tunica intima comprising endothelial cells, tunica media comprising smooth muscle cells, and tunica adventitia, where the condition or disease is associated with an incidence of cytolysis of the smooth muscle cells from beta-amyloid deposition in the cerebrovasculature that leads to formation of membrane attack complex of the complement system in the smooth muscle cells, where the substance comprises one or more than one inhibitor that causes inhibition of the formation of membrane attack complex of the complement system, and where the inhibition by the one or more than one inhibitor is sufficient to decrease the incidence of or to prevent the incidence of cytolysis of the smooth muscle cells. In one embodiment, the substance crosses the tunica intima and enters the tunica media. In another embodiment, the patient has a blood brain barrier comprising the tunica intima, the tunica media and the tunica adventitia, and the substance crosses the blood brain barrier.

In one embodiment, the one or more than one inhibitor is a plurality of inhibitors, such as for example both alphaGal lectin and human CD59 cDNA. In another embodiment, the plurality of inhibitors is two inhibitors. In another embodiment, the plurality of inhibitors is three inhibitors. In another embodiment, the plurality of inhibitors is four inhibitors. In one embodiment, the cerebral amyloid angiopathy related condition or disease is selected from the group consisting of one or more than one of Alzheimer's disease, a brain microbleed, cerebral amyloidosis of the parenchyma, mild cognitive impairment with amyloid plaques in the brain and a combination of the preceding. In a preferred embodiment, the one or more than one inhibitor specifically causes inhibition of the formation of membrane attack complex of the complement system in the tunica intima of the cerebrovasculature, tunica media of the cerebrovasculature, or both the tunica intima of the cerebrovasculature and tunica media of the cerebrovasculature, where specific inhibition of membrane attack complex is determined by a decrease of cell lysis after applying a predetermined amount of activated complement and the inhibitor as compared to amount of cell lysis with the same amount of activated complement without the inhibition. As will be understood by those with skill in the art with respect to this disclosure, specific inhibition can be measured either directly by increased light absorbance when the cells tested are erythrocytes or indirectly by increased light absorbance in proportion to overall metabolic activity in an MTT assay, or by any other suitable method according to techniques known to those with skill in the art.

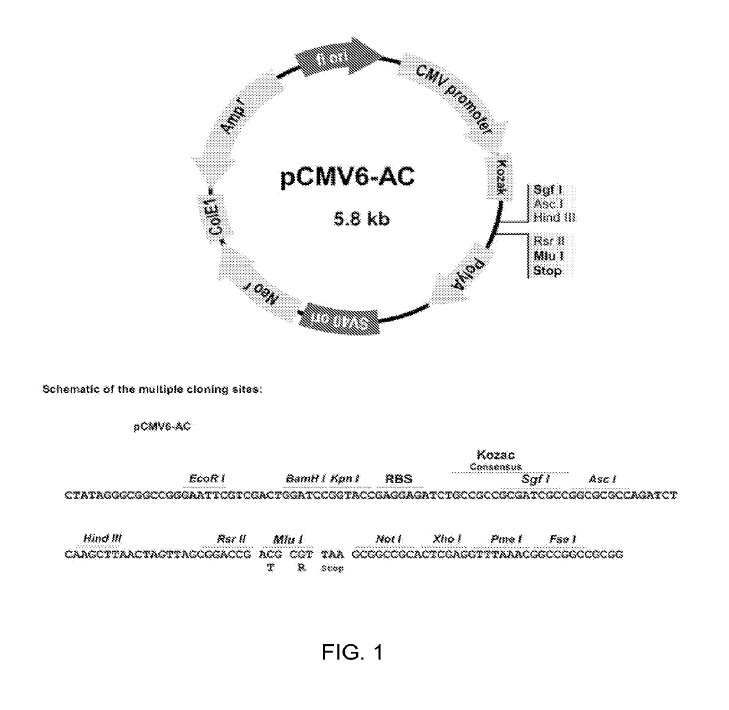

In one embodiment, one or more than one of the one or more than one inhibitor that causes inhibition of the formation of membrane attack complex of the complement system upregulates CD59 glycoprotein levels in the cerebrovasculature of the patient. In one embodiment, one or more than one of the one or more than one inhibitor that causes inhibition of the formation of membrane attack complex of the complement system is selected from the group consisting of one or more than one of alphaGal lectin (a lectin that binds to terminal Gal-alpha(1-3)Gal residues) which increases CD59 glycoprotein levels by induced ligation), anti-05 Mab (binds component 5 of the complement system thereby blocking component 5 from potentiating downstream complement molecules), C1-Inhibitor (prevents initiation of the complement cascade), factor H (accelerates decay of the C3 convertase), human CD59 cDNA (interrupts formation of the transmembrane pore in membrane attack complex), and a combination of the preceding. In a preferred embodiment, one of the one or more than one inhibitor that causes inhibition of the formation of membrane attack complex of the complement system is a plasmid comprising human CD59 cDNA. In a particularly preferred embodiment, one of the one or more than one inhibitor comprises human CD59 cDNA in the pCMV6-AC. Referring now to FIG. 1, there is shown a schematic depiction of one embodiment of the human CD59 cDNA in the pCMV6-AC that can be used as an inhibitor according to the present invention. As can be seen, without the CD59 cDNA, the pCMV6-AC plasmid is 5.8 kb. With the CD59 open reading frame (OriGene RC218343) the entire construct is 6.1 kb. The pCMV6-AC has the following key features for mammalian expression: cytomegalovirus (CMV) promoter (strong, constitutive, ubiquitous expression), and human growth hormone polyA (hGH polyA) signal sequence (leads to polyadenylation of the transcribed mRNA which is essential for mRNA stability in cells). The pCMV6-AC also comprises a modified bacterial origin of replication (allows for enhanced replication in E. coli to produce more plasmids), and a Neomycin phosphotransferase locus (confers Neomycin resistance and allows for selection of plasmid-containing bacteria or mammalian cells). As will be understood by those with skill in the art with respect to this disclosure, though the plasmid disclosed above and shown in FIG. 1 is suitable for the inhibitor according to the present invention, other plasmids comprising human CD59 gene that function as intended in the substance can also be used, such as for example the pCMV6-XL5 entry vector which is 4.7 kb that is identical to pCMV6-AC except that it lacks the Neomycin phosphotransferse locus and includes a T7 promoter for cell-free in vitro replication systems. As will be understood by those with skill in the art with respect to this disclosure, there are several commercially suitable splice variants of CD59 in plasmids suitable for use in the present invention.

In one embodiment, the substance for treating a cerebral amyloid angiopathy related condition or disease affecting cerebrovasculature in a patient further comprises one or more than one vehicle for transporting the one or more than one inhibitor that causes inhibition of the formation of membrane attack complex of the complement system into the cerebrovasculature. In one embodiment, one or more than one of the one or more than one vehicle is selected from the group consisting of chitosan nanoparticles, colloidal metallic nanoparticles, polymer nanoparticles and viral particles. In one embodiment, the one or more than one vehicle is a plurality of vehicles. In a preferred embodiment, the plurality of vehicles are chitosan nanoparticles having a maximum diameter of between 50-100 nm, and chitosan nanoparticles having a maximum diameter of between 200-400 nm. In a preferred embodiment, the plurality of vehicles are chitosan nanoparticles having a maximum diameter of between 50-100 nm, and chitosan nanoparticles having a maximum diameter of between 400-600 nm. In a preferred embodiment, the plurality of vehicles are chitosan nanoparticles having a maximum diameter of between 200-400 nm, and chitosan nanoparticles having a maximum diameter of between 400-600 nm. In a preferred embodiment, the plurality of vehicles are chitosan nanoparticles having a maximum diameter of between 50-100 nm, chitosan nanoparticles having a maximum diameter of between 200-400 nm, and chitosan nanoparticles having a maximum diameter of between 400-600 nm. In another embodiment, the plurality of vehicles is two vehicles. In another embodiment, the plurality of vehicles is three vehicles. In another embodiment, the plurality of vehicles is four vehicles. In a preferred embodiment, the one or more than one vehicle crosses the tunica intima and enters the tunica media. In another embodiment, the patient has a blood brain barrier comprising the tunica intima, the tunica media and the tunica adventitia, and the one or more than one vehicle crosses the blood brain barrier. In a preferred embodiment, at least one of the one or more than one vehicle comprises chitosan nanoparticles. Chitosan is a deacetylated product of chitin, a polysaccharide found in the internal structures and outer skeleton of some invertebrates including crabs, insects, lobsters and shrimps. Chitin is composed of β(1-4) linked units of the amino sugar N-acetyl-glucosamine linked, and is the main source for the production of chitosan. Medical grade chitosan (low protein, medium-high molecular weight) suitable for the present invention can be obtained from Scion Cardio-Vascular, Inc. Miami, Fla. US, though any suitable source can be used, as will be understood by those with skill in the art with respect to this disclosure. Chitosan nanoparticles are particularly suited as a vehicle according to the present invention as chitosan is a biologically well-tolerated and biodegradable, and the molecular properties of chitosan allow for easy encapsulation of the inhibitor by chitosan by readily available encapsulation techniques. Further chitosan nanoparticles have positive surface charges that allow for easy surface incorporation of the targeting agent for delivery of the substance to the cerebrovasculature, such as for example by using biotin/avidin conjugation methods, as will be understood by those with skill in the art with respect to this disclosure. Further, chitosan nanoparticles pass through the blood brain barrier due to the positive charges on the chitosan surface, thereby serving as an appropriate vehicle for delivery of the inhibitor to the cerebrovasculature according to the present invention. Additionally, chitosan nanoparticles without targeting agents are eliminated from the systemic circulation on the basis of particle size by either the kidney glomerular basement membrane for small nanoparticles or by degradation of larger nanoparticles in the liver and reticulo-endothelial system. Further, since chitosan has a structural similarity to sugars, chitosan acts as a cryo-protectant for proteins conjugated to the surface during lyophilization. Additionally, chitosan as the vehicle improves transfection efficiency when the inhibitor comprises a gene, such as for example transfection efficiency when the inhibitor is human CD59 cDNA. As will be understood by those with skill in the art with respect to this disclosure, though chitosan nanoparticles disclosed above are suitable for the vehicle according to the present invention, other vehicles that function as intended in the substance can also be used.

In one embodiment, the substance comprises one vehicle and a plurality of inhibitors, such as for example a substance comprising chitosan nanoparticles having a maximum diameter of between 200-400 nm as the vehicle and two or more than two inhibitors selected from the group consisting of alphaGal lectin, anti-05 Mab, C1-Inhibitor, factor H and human CD59 cDNA within the chitosan nanoparticles. In one embodiment, the substance comprises one vehicle and two inhibitors, such as for example a substance comprising chitosan nanoparticles having a maximum diameter of between 200-400 nm as the vehicle and alphaGal lectin and human CD59 cDNA as the inhibitors within the chitosan nanoparticles.

Chitosan nanoparticles as a vehicle according to the present invention can be made by any suitable method, as will be understood by those with skill in the art with respect to this disclosure. By way of example, in one embodiment, the vehicle is chitosan nanoparticles and the substance is produced as follows. Chitosan of a known molecular weight is dissolved (0.1% w/v) in 4.6 mM HCL and syringe filtered through 0.22 um to remove undissolved residues. After filtration, the pH of the chitosan solution is adjusted to pH 5 with 1 N NaOH. Tripolyphosphate (TPP) solution is prepared in ultra pure water and the pH corrected to pH 5. pCMV6 plasmids containing the human CD59 cDNA, alphaGal lectin, or any other inhibitor according to the present invention is added to the TPP solution at an appropriate concentration prior to mixing with the chitosan solution. While under magnetic stirring at 300 rpm, 2 ml of TPP solution is added to 14 ml of the chitosan solution. Nucleation of chitosan particles is spontaneous under these conditions. Stirring is continued for 45 minutes, after which the reaction is left undisturbed for 16 hours at room temperature. The resultant chitosan nanoparticles are 1000 nm (1 micron) or less in maximum diameter. The chitosan nanoparticles formed were 85% deacetylated and depyrogenated.

In one embodiment, the substance further comprises one or more than one targeting agent that recognizes the beta-amyloid deposited in the cerebrovasculature, where one or more than one targeting agent forms at least part of the surface of the substance when one or more than one targeting agent is combined with the inhibitor and the vehicle, thereby directing the substance to the beta-amyloid deposited in the cerebrovasculature when the substance is administered to the patient. In one embodiment, the targeting agent recognizes and attaches to a subset of conformationally unique beta-amyloid deposited between the cerebrovasculature, where the conformationally unique beta-amyloid deposited in the cerebrovasculature is specific for cerebral amyloid angiopathy. In another embodiment, when combined with the inhibitor and the vehicle, the targeting agent directs the substance to the beta-amyloid deposited in the cerebrovascular smooth muscle cells when the substance is administered to the patient. In one embodiment, at least one of the one or more than one targeting agent is a monoclonal antibody or is a fragment of a monoclonal antibody. In one embodiment, the targeting agent is selected from the group consisting of amyloid antibody (M31) Fab fragments (M31 monoclonal antibody is also known as rat anti M31/HP1; heterochromatin protein 1 homolog beta) (catalog number MBS214105, MyBioSource, Inc., San Diego, Calif., US), 6E10 beta amyloid monoclonal antibody (also known as Alzheimer disease amyloid protein; amyloid beta A protein; beta-amyloid peptide; cerebral vascular amyloid peptide peptidase; nexin-II; preA4; and protease nexin-II) (catalog number SIG-39300, Covance, Inc., San Diego, Calif., US), and a combination of the preceding, however, any targeting agent suitable for the purpose intended can be used as will be understood by those with skill in the art with respect to this disclosure. Amyloid antibody (M31) Fab fragments are rabbit monoclonal IgG specific for the subset of human and murine vascular amyloid beta assemblies that characterize cerebral amyloid angiopathy. In one embodiment, the one or more than one targeting agent is a plurality of targeting agents, such as for example both amyloid antibody (M31) Fab fragment and an antibody that recognized smooth muscle cells in the cerebrovasculature. In another embodiment, the plurality of targeting agents is two targeting agents. In another embodiment, the plurality of targeting agents is three targeting agents. In another embodiment, the plurality of targeting agents is four targeting agents.

The one or more than one targeting agent can be added to the one or more than one vehicle by any suitable method, as will be understood by those with skill in the art with respect to this disclosure. In one embodiment, the substance comprises an inhibitor, a vehicle and a targeting agent, and making the substance comprises producing the combination of the inhibitor and vehicle, and then adding the targeting agent to the combination by conjugation using a biotin-streptavidin interaction. For example, streptavidin conjugation to amyloid antibody (M31) Fab fragments is accomplished by labeling with the EasyLink Streptavidin Conjugation Kit (Abcam, Cambridge, Mass., US) according to the manufacturers instructions. Purified Fab fragments are incubated with the modifier and conjugate solution for 3 hours. After quenching the reaction for 30 minutes, the Fab fragments conjugated to avidin are mixed with biotinylated chitosan nanoparticles. Biotinylation of chitosan is accomplished prior to precipitation of chitosan nanoparticles. SulfoNHS-LC-Biotin (Thermo Fisher Scientific Inc., Rockford, Ill., US) is dissolved in PBS and incubated with chitosan flake (0.1% w/v) at room temperature for 3 hours. Free SulfoNHS-LC-Biotin is removed by dialysis. The degree of biotinylation is determined by 2-(4-hydroxyazobenzene)benzoic acid (HABA) assay, with decreases in absorption recorded at 500 nm. However, making the substance comprising the targeting agents can comprise any suitable method, as will be understood by those with skill in the art with respect to this disclosure.

In one embodiment, the substance further comprises one or more than one additional chemical that enables determination of plasmid transfection efficacy where the inhibitor is a plasmid, or enables tracking of the substance. In one embodiment, at least one of the one or more than one additional chemical is selected from the group consisting of hydroxycoumarin and green fluorescent protein. As will be understood by those with skill in the art with respect to this disclosure, hydroxycoumarin and green fluorescent protein can be added to the substance according to techniques known to those with skill in the art, such as adding hydroxycoumarin during precipitation of the chitosan nanoparticles which incorporates the hydroxycoumarin into the structure of the nanoparticles, and chemically modifying green fluorescent protein to link it with streptavidin, which is then introduced to biotinylated nanoparticles formation of the biotinylated nanoparticles.

According to another embodiment of the present invention, there is provided a pharmaceutical for treating a cerebral amyloid angiopathy related condition or disease. The pharmaceutical comprises one or more than one substance according to the present invention. In one embodiment, the pharmaceutical further comprises one or more than one of a binder, a buffer, a coloring chemical, a flavoring chemical and a preservative, as will be understood by those with skill in the art with respect to this disclosure.

According to another embodiment of the present invention, there is provided a method for treating a cerebral amyloid angiopathy related condition or disease affecting cerebrovasculature in a patient, where the cerebrovasculature comprises tunica intima comprising endothelial cells, tunica media comprising smooth muscle cells, and tunica adventitia, where the condition or disease is associated with an incidence of cytolysis of the smooth muscle cells from beta-amyloid deposition in the cerebrovasculature that leads to formation of membrane attack complex of the complement system in the smooth muscle cells. The method comprises, first, identifying a patient with a cerebral amyloid angiopathy related condition or disease suitable for treatment by the present method. In a preferred embodiment, the patient is a human. In one embodiment, the cerebral amyloid angiopathy related condition or disease is selected from the group consisting of one or more than one of Alzheimer's disease, a brain microbleed, cerebral amyloidosis of the parenchyma, mild cognitive impairment with amyloid plaques in the brain and a combination of the preceding. In another embodiment, identifying the patient comprises consulting patient records to determine if the patient has a cerebral amyloid angiopathy related condition or disease suitable for treatment by the present method. In another embodiment, identifying the patient comprises diagnosing the patient with a cerebral amyloid angiopathy related condition or disease suitable for treatment by the present method. In one embodiment, diagnosing the patient comprises performing one or more than one of action selected from the group consisting of identifying one or more than one marker for cerebral amyloid angiopathy in blood or another body fluid of the patient, performing cognitive testing, performing an invasive procedure (such as for example biopsying brain tissue of the patient), performing a non-invasive imaging procedure (such as for example computerized tomography, magnetic resonance imaging or ultrasound), and performing a physical examination.

Next, the method comprises providing one or more than one substance that comprises an inhibitor that causes inhibition of the formation of membrane attack complex of the complement system, or comprises providing one or more than one pharmaceutical comprising one or more than one substance that comprises an inhibitor that causes inhibition of the formation of membrane attack complex of the complement system, or comprises providing both one or more than one substance that comprises an inhibitor that causes inhibition of the formation of membrane attack complex of the complement system and one or more than one pharmaceutical comprising one or more than one substance that comprises an inhibitor that causes inhibition of the formation of membrane attack complex of the complement system, where the inhibition by the inhibitor is sufficient to decrease the incidence of or to prevent the incidence of cytolysis of the smooth muscle cells. In one embodiment, one or more than one of the one or more than one substance is a substance according to the present invention. In another embodiment, one or more than one of the one or more than one pharmaceutical is a pharmaceutical according to the present invention. In one embodiment, the one or more than one substance is a plurality of substances. In one embodiment, the one or more than one substance is two substances, such as for example chitosan nanoparticles as a vehicle comprising alphaGal lectin as an inhibitor, and chitosan nanoparticles as a vehicle comprising human CD59 cDNA as an inhibitor. In one embodiment, the one or more than one pharmaceutical is a plurality of pharmaceuticals. In one embodiment, the one or more than one pharmaceutical is two pharmaceuticals.

Then, the method comprises administering one or more than one dose of the one or more than one substance or administering one or more than one dose of the one or more than one pharmaceutical to the patient by a route. In one embodiment, the one or more than one dose is one dose. In another embodiment, the one or more than one dose is a plurality of doses. In one embodiment, the plurality of doses is two doses. In another embodiment, the plurality of doses is three doses. In another embodiment, the plurality of doses is four doses. In another embodiment, the plurality of doses is more than four doses. In one embodiment, the one or more than one dose is administered daily for a predetermined amount of time. In another embodiment, the one or more than one dose is administered twice daily for a predetermined amount of time. In another embodiment, the one or more than one dose is administered weekly for a predetermined amount of time. In another embodiment, the one or more than one dose is administered monthly for a predetermined amount of time. In another embodiment, the one or more than one dose is administered between once a day and once a week for a predetermined amount of time. In another embodiment, the one or more than one dose is administered between once a day and once a month for a predetermined amount of time. In one embodiment, the dose is between 0.000001 mg/m² body surface area and 100 g/m² body surface area. In another embodiment, the dose is between 0.0001 mg/m² body surface area and 10 g/m² body surface area. In another embodiment, the dose is between 1 mg/m² body surface area and 1 g/m² body surface area. In one embodiment, the route is selected from the group consisting of intraarterial injection, intramuscular injection, intranasal spray and intravenous injection.

In one embodiment, the method further comprises determining the effect of treatment on the patient. In one embodiment, determining the effect of treatment on the patient comprises performing one or more than one of action selected from the group consisting of identifying one or more than one marker for cerebral amyloid angiopathy in blood or another body fluid of the patient, performing cognitive testing, performing an invasive procedure (such as for example biopsying brain tissue of the patient), performing a non-invasive imaging procedure (such as for example computerized tomography, magnetic resonance imaging or ultrasound), and performing a physical examination. In another embodiment, the method further comprises adjusting treatment. In one embodiment, adjusting treatment comprising administering to the patient one or more than one additional dose of the one or more than one substance or administering one or more than one additional dose of the one or more than one pharmaceutical to the patient.

### Example I

### Determination of the Mechanism of Damage in Cerebral Amyloid Angiopathy

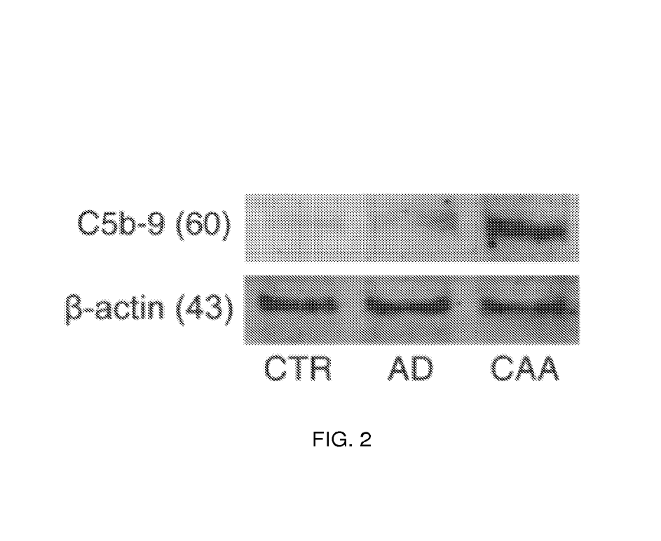

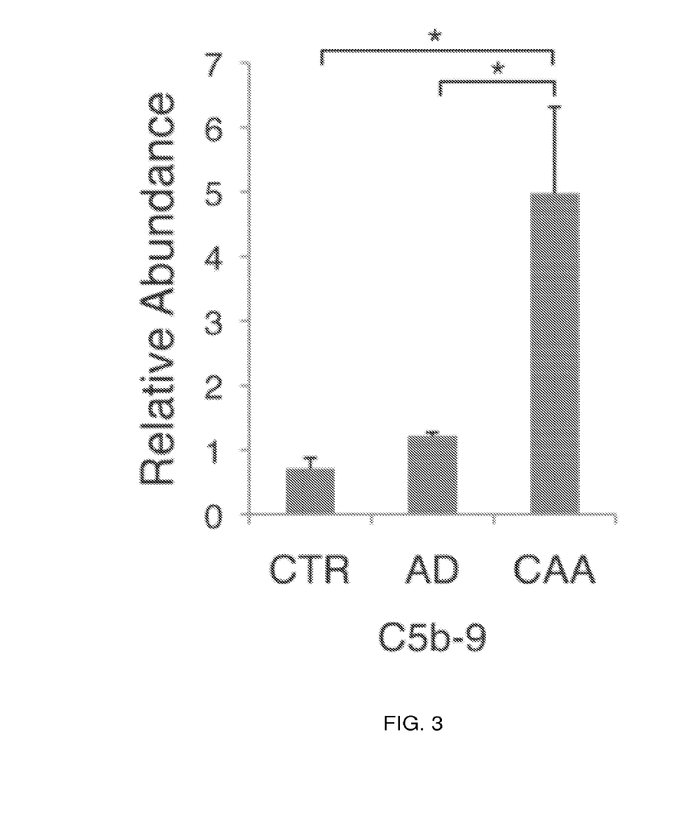

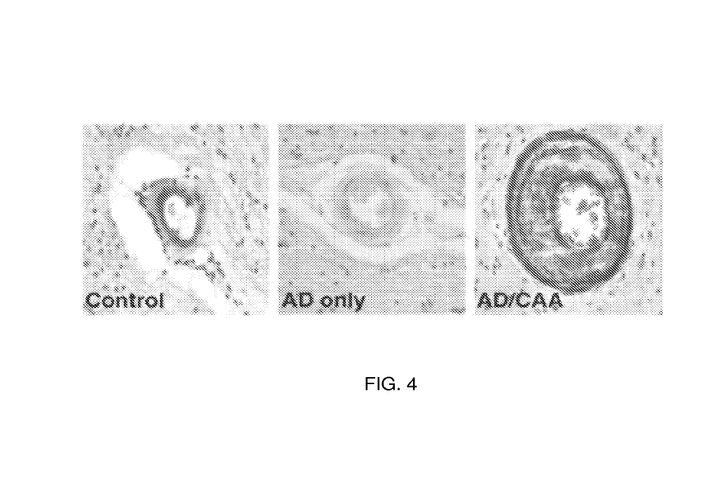

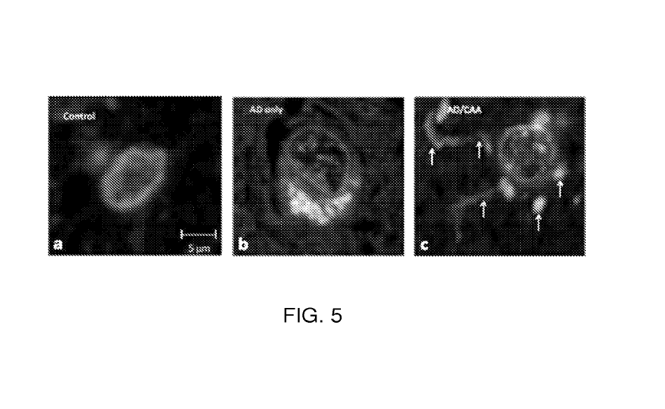

To determine the mechanism of damage in cerebral amyloid angiopathy, a series of studies was performed on blood vessels from frozen postmortem occipital lobe sections that were sonicated on ice, and then probed with antibodies. Referring now to FIG. 2, FIG. 3, FIG. 4 and FIG. 5, there are shown, respectively, western blots showing the accumulation of C6 (upper) and beta-actin (a protein expressed in all cells and used as a loading control to normalize C6 values) (lower) in blood vessel walls from control brain tissue (left), in blood vessel walls from brain tissue from patients diagnosed with Alzheimer's disease (AD, center), and in blood vessel walls from brain tissue from patients diagnosed with Alzheimer's disease concurrent with cerebral amyloid angiopathy (AD/CAA, right) (FIG. 2); a histogram showing the average concentration of C6 from multiple western blots (n=4) in blood vessel walls from control brain tissue, in blood vessel walls from brain tissue from patients diagnosed with Alzheimer's disease (AD), and in blood vessel walls from brain tissue from patients diagnosed with Alzheimer's disease concurrent with cerebral amyloid angiopathy (AD/CAA) (FIG. 3); photomicrographs showing C6 localization in the walls of blood vessels from control brain tissue (left), in the walls of blood vessels from brain tissue from patients diagnosed with Alzheimer's disease (AD, center), and in the walls of blood vessels from brain tissue from patients diagnosed with Alzheimer's disease concurrent with cerebral amyloid angiopathy (AD/CAA, right) by immunohisto-chemistry with DAB-staining (FIG. 4); and confocal micrographs of microglia from control brain tissue (A, left), microglia from brain tissue from patients diagnosed with Alzheimer's disease (B, center), and microglia from brain tissue from patients diagnosed with Alzheimer's disease concurrent with cerebral amyloid angiopathy (C, right), where the brain tissue was stained for alpha2-macroglobulin (a2M) and beta-amyloid (A, B), and for beta-amyloid and CD11b (C) (FIG. 5). As can be seen in FIG. 2 and FIG. 3, C6 accumulated more extensively in blood vessel walls of brain tissue from patients diagnosed with Alzheimer's disease concurrent with cerebral amyloid angiopathy than in blood vessel walls of control brain tissue or brain tissue from patients diagnosed with Alzheimer's disease alone. No such pattern was detected for the localization of CD11b. Further, CD11b with beta-amyloid show punctate colocalization at the plasma membrane of microglia from brain tissue from patients diagnosed with Alzheimer's disease concurrent with cerebral amyloid angiopathy as indicated by the arrows (FIG. 5, right), rather than the internalized phagocytic vesicles seen in normal beta-amyloid trafficking of brain tissue from patients diagnosed with Alzheimer's disease alone (FIG. 5, center), where control brain tissue show only slight colocalization for CD11b and beta-amyloid (FIG. 5, left), thereby demonstrating that beta-amyloid is taken up normally via the alpha2-macroglobulin receptor in patients diagnosed with Alzheimer's disease without concurrent cerebral amyloid angiopathy, but is associated with the cell surface receptor CD11b in the brains of patients diagnosed with Alzheimer's disease concurrent with cerebral amyloid angiopathy. CD11b is known to bind C3b, the molecule at the crossroads of the complement cascade.

These studies demonstrated that the mechanism by which aging microglia in cerebral amyloid angiopathy-damaged brains remove beta-amyloid from cerebral tissues changes from the established alpha2-macroglobulin/LRP-mediated clearance of beta-amyloid (endophagocytosis) to CD11b/C3b receptor-mediated shuttling on the microglial surface (opsonization). The CD11b/C3b receptor/beta-amyloid complex is delivered by microglia to the abluminal vascular wall for disposal of beta-amyloid into the circulatory system. This perpetuates cerebrovascular injury because C3b initiates the complement cascade leading to formation of the membrane attack complex (MAC, C5b-C9), the transmembrane pore part of the innate immune reaction that is the terminal lytic component of the cascade. Membrane attack complex weakens the vessel by destroying cerebrovascular smooth muscle cells (SMC) resulting in vessel fragility, disruption of the blood-brain barrier (BLOOD BRAIN BARRIER) and increasing the probability of brain microbleeds (BMB).

Therefore, these studies identified a treatable pathogenic mechanism for cerebral amyloid angiopathy, namely, inhibiting the formation of membrane attack complex in the cerebrovasculature. In one embodiment, inhibiting the formation of membrane attack complex in the cerebrovasculature, and thus inhibiting induced cytotoxicity in cerebrovascular smooth muscle cells, is accomplished by upregulating levels of CD59 glycoprotein.

### Example II

### Method for Treating a Cerebral Amyloid Angiopathy in a Patient

According to one embodiment of the present invention, a patient with cerebral amyloid angiopathy is treated as follows. First, a patient is identified with Alzheimer's disease associated with cerebral amyloid angiopathy by cognitive analysis followed by brain biopsy. Next, a substance according to the present invention is provided that comprises human CD59 cDNA in the pCMV6-AC as the inhibitor, chitosan nanoparticles as the vehicle and amyloid antibody (M31) Fab fragments as the targeting agent. Then, the substance is administered to the patient once per week for five weeks at a dose of 1 mg/m2 body surface area, and the once a month thereafter.

The substance binds to the beta-amyloid deposition in the cerebrovasculature. The substance is then endocytosed by cerebrovascular smooth muscle cells and transported by the endosomal/lysosomal pathway to liberate the CD59 cDNA. The CD59 cDNA is processed into mRNA, and the mRNA is transcribed, upregulating CD59 glycoprotein levels. The CD59 blocks the addition of complement component C9 to C5b-8, effectively inhibiting formation of membrane attack complex and decreasing or preventing cytolysis of the smooth muscle cells, and thereby treating the cerebral amyloid angiopathy.

Although the present invention has been discussed in considerable detail with reference to certain preferred embodiments, other embodiments are possible. Therefore, the scope of the appended claims should not be limited to the description of preferred embodiments contained in this disclosure.

1. A substance for treating a cerebral amyloid angiopathy related condition or disease affecting cerebrovasculature in a patient, where the cerebrovasculature comprises tunica intima comprising endothelial cells, tunica media comprising smooth muscle cells, and tunica adventitia, where the condition or disease is associated with an incidence of cytolysis of the smooth muscle cells from beta-amyloid deposition in the cerebrovasculature that leads to formation of membrane attack complex of the complement system in the smooth muscle cells, the substance comprising:
one or more than one inhibitor that causes inhibition of the formation of membrane attack complex of the complement system; and
one or more than one vehicle for transporting the one or more than one inhibitor into the cerebrovasculature;
where the inhibition by the inhibitor is sufficient to decrease the incidence of or to prevent the incidence of cytolysis of the smooth muscle cells.

2. The substance of claim 1, where the substance crosses the tunica intima and enters the tunica media.

3. The substance of claim 1, where the patient has a blood brain barrier comprising the tunica intima, the tunica media and the tunica adventitia, and where the substance crosses the blood brain barrier.

4-6. (canceled)

7. The substance of claim 1, where the one or more inhibitors is a plurality of inhibitors, where the plurality of inhibitors is four inhibitors.

8. The substance of claim 1, where the cerebral amyloid angiopathy related condition or disease is selected from the group consisting of one or more than one of Alzheimer's disease, a brain microbleed, cerebral amyloidosis of the parenchyma, mild cognitive impairment with amyloid plaques in the brain and a combination of the preceding.

9. The substance of claim 1, where the one or more than one inhibitor specifically causes inhibition of the formation of membrane attack complex of the complement system in the tunica intima of the cerebrovasculature, tunica media of the cerebrovasculature, or both the tunica intima of the cerebrovasculature and tunica media of the cerebrovasculature.

10. (canceled)

11. The substance of claim 1, where one or more than one of the one or more than one inhibitor that causes inhibition of the formation of membrane attack complex of the complement system is selected from the group consisting of one or more than one of alpha Gal lectin, anti-05 Mab, C1-Inhibitor, factor H, human CD59 cDNA, and a combination of the preceding.

12. The substance of claim 1, where one of the one or more than one inhibitor that causes inhibition of the formation of membrane attack complex of the complement system is a plasmid comprising human CD59 cDNA.

13. the substance of claim 12, where one of the one or more than one inhibitor comprises human CD59 cDNA in the pCMV6-AC plasmid, or human CD59 cDNA in the pCMV6-XL5 plasmid.

14-18. (canceled)

19. The substance of claim 1, where at least one of the one or more than one vehicle comprises chitosan nanoparticles.

20-21. (canceled)

22. The substance of claim 1, further comprising one or more than one targeting agent that recognizes the beta-amyloid deposited in the cerebrovasculature, where one or more than one targeting agent forms at least part of the surface of the substance when one or more than one targeting agent is combined with the inhibitor and the vehicle, thereby directing the substance to the beta-amyloid deposited in the cerebrovasculature when the substance is administered to the patient.

23. The substance of claim 22, where the targeting agent recognizes and attaches to a subset of conformationally unique beta-amyloid deposited in the cerebrovasculature, where the conformationally unique beta-amyloid deposited in the cerebrovasculature is specific for cerebral amyloid angiopathy.

24. The substance of claim 22, where when combined with the inhibitor and the vehicle, the targeting agent directs the substance to the beta-amyloid deposited in the cerebrovascular smooth muscle cells when the substance is administered to the patient.

25. The substance of claim 22, where at least one of the one or more than one targeting agent is a monoclonal antibody or is a fragment of a monoclonal antibody.

26. The substance of claim 22, where at least one of the one or more than one targeting agent is selected from the group consisting of amyloid antibody (M31) Fab fragments, 6E10 beta amyloid monoclonal antibody and a combination of the preceding.

27-29. (canceled)

30. The substance of claim 22, where the one or more than one targeting agents is a plurality of targeting agents, where the plurality of targeting agents is four targeting agents.

31. The substance of claim 1, where the substance further comprises one or more than one additional chemical that enables determination of plasmid transfection efficacy where the inhibitor is a plasmid, or enables tracking of the substance.

32. The substance of claim 31, where at least one of the one or more than one additional chemical is selected from the group consisting of hydroxycoumarin and green fluorescent protein.

33. A pharmaceutical for treating a cerebral amyloid angiopathy related condition or disease, the pharmaceutical comprising one or more than one substance according to claim 1, and further comprising one or more than one of a binder, a buffer, a coloring chemical, a flavoring chemical and a preservative.

34. A method for treating a cerebral amyloid angiopathy related condition or disease affecting cerebrovasculature in a patient, where the cerebrovasculature comprises tunica intima comprising endothelial cells, tunica media comprising smooth muscle cells, and tunica adventitia, where the condition or disease is associated with an incidence of cytolysis of the smooth muscle cells from beta-amyloid deposition in the cerebrovasculature that leads to formation of membrane attack complex of the complement system in the smooth muscle cells, the method comprising:
a) identifying a patient with a cerebral amyloid angiopathy related condition or disease suitable for treatment;
b) providing one or more than one substance that comprises an inhibitor that causes inhibition of the formation of membrane attack complex of the complement system, or comprises providing one or more than one pharmaceutical comprising one or more than one substance that comprises an inhibitor that causes inhibition of the formation of membrane attack complex of the complement system, or comprises providing both one or more than one substance that comprises an inhibitor that causes inhibition of the formation of membrane attack complex of the complement system and one or more than one pharmaceutical comprising one or more than one substance that comprises an inhibitor that causes inhibition of the formation of membrane attack complex of the complement system, where the inhibition by the inhibitor is sufficient to decrease the incidence of or to prevent the incidence of cytolysis of the smooth muscle cells; and
c) administering one or more than one dose of the one or more than one substance or administering one or more than one dose of the one or more than one pharmaceutical to the patient by a route.

35. (canceled)

36. The method of claim 34, where the cerebral amyloid angiopathy related condition or disease is selected from the group consisting of one or more than one of Alzheimer's disease, a brain microbleed, cerebral amyloidosis of the parenchyma, mild cognitive impairment with amyloid plaques in the brain and a combination of the preceding.

37. The method of claim 34, where identifying the patient comprises consulting patient records to determine if the patient has a cerebral amyloid angiopathy related condition or disease suitable for treatment.

38. The method of claim 34, where identifying the patient comprises diagnosing the patient with a cerebral amyloid angiopathy related condition or disease suitable for treatment.

39. The method of claim 38, where diagnosing the patient comprises performing one or more than one of action selected from the group consisting of identifying one or more than one marker for cerebral amyloid angiopathy in blood or another body fluid of the patient, performing cognitive testing, performing an invasive procedure, performing a non-invasive imaging procedure, and performing a physical examination.

40-99. (canceled)


SEQUENCE LISTING (1 sequence)

SEQ ID NO: 1 - DNA, 147 bp, Artificial Sequence
  Multiple cloning site
ctatagggcggccgggaattcgtcgactggatccggtaccgaggagatctgccgccgcgatcgccggcgcgccagatctcaagcttaactagttagcggaccgacgcgttaagcggccgcactcgaggtttaaacggccggccgcgg